(19) United States
(12) Patent Application Publication
(10) Pub. No.: US 2008/0015252 A1
(43) Pub. Date: Jan. 17, 2008

(54) THERAPEUTIC AGENT FOR VIRUS-ASSOCIATED MALIGNANCY

(75) Inventor: Naoki MORI, Naha-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: TROPICAL TECHNOLOGY CENTER Ltd., Uruma-shi (JP); UNIVERSITY OF THE RYUKYUS, Nakagami-gun (JP)

(21) Appl. No.: 11/681,488

(22) Filed: Mar. 2, 2007

(30) Foreign Application Priority Data
Jul. 11, 2006 (JP) 2006-190076

(51) Int. Cl. A61K 31/336; A61P 35/00; A61P 35/02

(52) U.S. Cl. 514/475

(57) ABSTRACT

A therapeutic agent comprising fucoxanthin or fucoxanthinol as an active component is disclosed. The therapeutic agent is effective and high clinical utility for medical treatment and prevention of virus-associated malignancy such as adult T-cell leukemia and Burkitt lymphoma.

### TECHNICAL FIELD

The present invention relates to a therapeutic agent for virus-associated malignancy and, more particularly, to a therapeutic agent for virus-associated malignancy effective for treating or preventing malignancy caused by viral infection such as adult T-cell leukemia and Burkitt's lymphoma.

### DESCRIPTION OF BACKGROUND ART

In the present specification, virus-associated malignancy indicates a malignancy caused by viral infection as an origin such as adult T-cell leukemia (hereinafter referred to as “ATL”) and Burkitt's lymphoma (hereinafter referred to as “BL”).

Among these, ATL is a poor-prognosis leukemia/limphoma originating from a CD4± T-lymph cell induced by infection with human T-cell leukemia virus type-I (hereinafter referred to as “HTLV-I”), which is a human retrovirus. The number of HTLV-I-infected persons (HTLV-I carriers) is presumed to be about 1,200,000 in Japan (about 1% of the population). There is a local specificity to the number of HTLV-I carriers. In Japan, there are many HTLV-I carriers in the southwest district (Kyushu, Okinawa, and Shikoku). Worldwide, there are many HTLV-I carriers in tropical areas such as the Caribbean area and South America.

Many leukemia cells originating from ATL have a phenotype of CD4 positive and CD8 negative helper T-cells and are accompanied by unique nuclear denaturing. HTLV-I which is a C-type retrovirus and was called ATLV at the beginning, has been identified to be a causative virus of ATL, and epidemiological investigations have revealed that HTLV-I carriers have not only increased incidence of ATL, but also increased incidence of malignancy in general, and that nervous system disease called HAM (HTLV-I-associated myelopathy) and immunological diseases are induced in HTLV-I carriers. In this manner, HTLV-I is revealed as being related not only to ATL but also to other diseases.

Moreover, the involvement of HTLV-I in rheumatism-like chronic arthritis, Sjogren's syndrome, uveitis of the eyes, and the like has been indicated. ATL induced by infection with HTLV-I rapidly becomes serious once developed and its medical treatment is extremely difficult. Chemotherapy or radiotherapy applicable to malignant lymphoma has conventionally been used for treating ATL. However, they are a temporary symptomatic treatment and not an essential treating method.

On the other hand, Burkitt lymphoma (BL) is a blood tumor of which one of the causes is infection with Epstein-Barr virus (hereinafter referred to as “EBV”) which is a kind of herpesviruses. There are many EBV-related BLs among opportunistic lymphoma induced in patients with an impaired immune system and such BLs also exhibit a poor-prognosis. EBV is also a causative virus inducing infectious mononucleosis among young adults and is known to be related to nasopharyngeal carcinoma, some stomach cancers, and Hodgkin's disease.

However, there is no medicine having a high clinical efficiency and high safety for treating these virus-associated malignancy caused by viral infection. Development of a novel medicine has been strongly desired.

### DISCLOSURE OF THE INVENTION

### Problems to be Solved by the Invention

Therefore, an object of the present invention is to discover a substrate effective for medical treatment and prevention of virus-associated malignancy and to develop a therapeutic agent for virus-associated malignancy with high clinical utility using the substrate.

### MEANS FOR SOLVING THE PROBLEM

The present inventors have undertaken research on the subject of HTLV-1 and EBV and investigated medicines clinically useful for treating ATL and BL for a long period of time. As a result, the present inventors have found that fucoxanthin and fucoxanthinol contained in seaweeds act specifically on virus-associated malignancy and can attain the objective of functioning as a curative medicine. This finding has led to the completion of the present invention.

Accordingly, the present invention provides a therapeutic agent for virus-associated malignancy comprising fucoxanthin or fucoxanthinol as an active component.

In addition, the present invention provides a method of treating virus-associated malignancy comprising administering fucoxanthin or fucoxanthinol to a patient having virus-associated malignancy.

The present invention further provides use of fucoxanthin or fucoxanthinol in manufacture of a therapeutic agent for virus-associated malignancy.

### EFFECT OF THE INVENTION

Fucoxanthin and fucoxanthinol, which are active components of the therapeutic agent for virus-associated malignancy of the present invention (hereinafter referred to as “malignancy treating agent”), selectively act on HTLV-1-infected cells and ATL cells, but substantially do not act on normal cells. Specifically, the concentration of fucoxanthin or fucoxanthinol causing apoptosis in these cells is significantly lower than the corresponding concentration of β-carotene or astaxanthin. Especially, fucoxanthinol causes apoptosis at a concentration of one micromole or less and the survival rate of the above-mentioned cells is almost zero.

Since fucoxanthin and fucoxanthinol cause apoptosis specifically in virus-associated malignancy at a significantly high rate, a malignancy treating agent containing fucoxanthin or fucoxanthinol as an active component is very useful for treating or preventing malignancy generated by viral infection such as ATL or BL.

### BEST MODE FOR CARRYING OUT THE INVENTION

The active component of the malignancy treating agent of the present invention is fucoxanthin or fucoxanthinol. Among these, fucoxanthin is a compound contained in seaweeds which we take routinely, for example, brown algae such as Undaria pinnatifida, Laminaria, and Hedwigiaceae. Fucoxanthin is a carotenoid with low toxicity shown by the following formula (I). Fucoxanthinol is a deacetylation compound obtained by hydrolysis of fucoxanthin and is shown by the following formula (II).

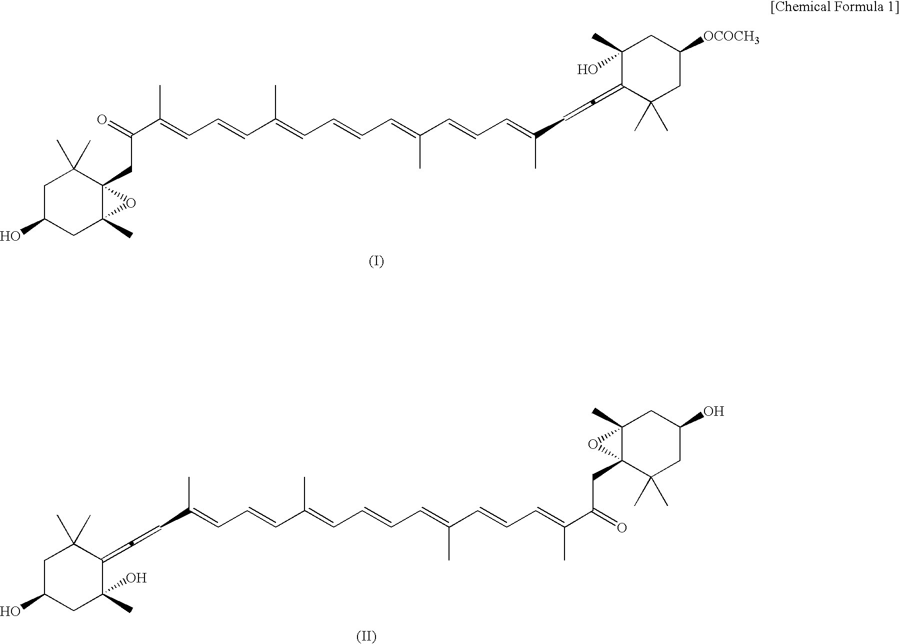

Fucoxanthin has been known to exhibit growth-suppressing activity of neuroblastoma, prostatic cancer, malignant melanoma, colorectal cancer, and acute promyelocytic leukemia cells and to exhibit carcinogenic retardation activity for colorectal cancer and a duodenum tumor in vitro. However, fucoxanthin is not known to exhibit outstanding antitumor activity against other cancers, for example, virus-associated malignancy such as virus-related leukemia and lymphoma. Furthermore, fucoxanthinol which is a deacetylation compound of fucoxanthin has not been known to exhibit a more excellent antitumor activity against virus-associated malignancy as compared with fucoxanthin.

Thus, the fact that fucoxanthin and fucoxanthinol can be effectively used as an agent against virus-associated malignancy for preventing and treating ATL and BL has been discovered for the first time by the present inventors.

As mentioned above, fucoxanthin is a compound contained in brown algae such as Undaria pinnatifida, Laminaria, and Hedwigiaceae. As an example of fucoxanthin preferably used in the present invention, a refined fucoxanthin product obtained by dipping brown algae such as seaweeds of Sargassum fulvellum or dry Underaia pinnatifida in an organic solvent such as methanol or acetone for about 18 hours at room temperature under shaded conditions to obtain an extract, condensing the extract, and separating fucoxanthin by liquid chromatography such as Diaion HP20™ (manufactured by Mitsubishi Chemical Corp.), Toyopearl HW40F™ (manufactured by Tosoh Corp.), or ODS (Wakogel 50C18™ manufactured by Wako Pure Chemical Industries, Ltd.), and refining the fucoxanthin by repeating recrystallization can be given.

Fucoxanthinol is a deacetylation compound obtained by hydrolysis of fucoxanthin and can be obtained by, for example, reacting various hydrolases with fucoxanthin.

More specifically, as an example of the fucoxanthinol used in the present invention, fucoxanthinol obtained by a method partly modifying a lipase decomposition method described by T. Matsuno, M. Ookubo, T. Nishizawa, and I. Shimizu (Chem. Pharm. Bull., 32, 4309-4315 (1984)) can be given.

The above-mentioned fucoxanthin or fucoxanthinol can be used as an active component of the malignancy treating agent of the present invention after purifying by a known purification method, if necessary.

Specifically, in order to prepare the malignancy treating agent of the present invention, fucoxanthin or fucoxanthinol (hereinafter referred to from time to time as “fucoxanthins”) may be combined with known drug carriers, as required.

The malignancy treating agent can be prepared into orally administered forms such as tablets, capsules, a powder preparation, granules, a liquid preparation, and a syrup, or parenterally administered forms such as an injection, an agent for an intravenous drip, a drug for external application, a suppository, and a pasting agent.

As examples of the drug carrier which can be used for preparing these preparations, known carriers for solid preparations, including vehicles such as starch, lactose, sucrose, mannitol, corn starch, microcrystalline cellulose, carboxymethylcellulose, and silicic-acid sugar; binders such as polyvinyl alcohol, polyvinyl pyrrolidone, polyvinyl ether, ethyl cellulose, gum arabic, traganth, gelatin, hydroxypropyl cellulose, dextrin, and pectin; lubricants such as magnesium stearate, talc, and polyethylene glycol; disintegrators; disintegrator adjutants; stabilizers; and carriers for liquid preparations, including liquid components such as water, ethyl alcohol, ethylene glycol, and glycerol; surfactants such as polyoxyethylene sorbitan fatty acid ester; taste components such as glucose and amino acid; solubilizing agents; colorants; and preservatives can be given. For forming preparations for external application, suppositories, and pasting agents, carriers known in the art conforming to respective forms can be used.

The amount of the fucoxanthins blended with the malignancy treating agent of the present invention varies according to the type of the disease, the degree of the disease, the age of the patient, and the like. For example, in the case of an oral preparation in which fucoxanthin is an active component, a preferable daily dosage for an adult is from about 0.1 mg to 300 mg, and about 1/10 of that amount in the case of intravascular injection.

In the case of an oral preparation in which fucoxanthinol is an active component, a preferable daily dosage for an adult is from about 0.05 mg to 100 mg, and about 1/10 of that amount in the case of intravascular injection.

### EXAMPLES

The present invention will be described in more detail by way of Reference Examples and Examples which should not be construed as limiting the present invention.

### Reference Example 1

### (Preparation of Fucoxanthin)

3.15 kg of dry Sargassum fulvellum was cut into pieces and extracted twice using 20 L of methanol at room temperature for 18 hours. The extract was concentrated to 1 L and partitioned twice using 800 mL of hexane each time. The methanol layer was concentrated. The concentrate was added to a HP20 column (φ55×150 mm) and eluted with 1.5 L of methanol and 600 mL of acetone. The fraction eluted with methanol was concentrated. The concentrate was added to a HW40F column (φ30×500 mm) and eluted with methanol. A fucoxanthin fraction was concentrated and the concentrate was recrystallized from 90% methanol twice to obtain 200 mg of purified fucoxanthin. The fucoxanthin purity was confirmed to be 95% or more by HPLC and a ¹H-NMR spectrum and the chemical structure was confirmed by NMR and MS spectra. The resulting purified fucoxanthin was used in the following Examples.

### Reference Example 2

### (Preparation of Fucoxanthinol)

100 mg of the purified fucoxanthin obtained by Reference Example 1 was dissolved in 2 mL of acetone. On the other hand. 2 g of Candidarugosa origin-lipase (manufactured by Sigma) was used as the lipase and dissolved in 22.5 mL of a 0.1 M phosphate buffer solution (pH 7.0). Both solutions were mixed and the mixture was heated at 37° C. for 18 hours. The reaction solution was filtered and the solvent was removed. The residue was extracted with 50 mL of acetone to collect a fucoxanthin reaction product. The reaction product was again subjected to the above lipase reaction. The resulting fucoxanthin reaction product was separated and purified by HPLC (Cosmosil ODS 5C18-AR-II20×250 mm, 80% MeOH, 5 mL/min, manufactured by Nacalai Tesque, Inc.) to obtain purified fucoxanthinol. The fucoxanthinol purity was confirmed to be 95% or more by HPLC and a ¹H-NMR spectrum and the chemical structure was confirmed by NMR and MS spectra. The resulting purified fucoxanthinol was used in the following Examples.

### Example 1

### Measurement of Proliferation Potency of Viral-Infected Cell Strains

### (Method)

Cells of HTLV-I-infected T-lymph cell lines (MT-2, MT-4, HUT-102. ED-40515 (−)) EBV-infected B cell lines (Raji. Daudi, B95-8/BJAB. B95-8/Ramos, LCL-Ka, LCL-Ku), a cervical cancer cell line (HeLa), and a chronic myeloid leukemia cell line (K-562), each adjusted to a concentration of 2×10⁵ cells/mL with an RPMI 1640 culture medium containing 10% fetal bovine serum, were spread over a 96-well plate in an amount of 1×10⁴ cells/well.

Next, 50 μL/well of fucoxanthin, fucoxanthinol, β-carotene, and astaxanthin were added to make final concentrations of 10, 5, 2.5, 1.25, and 0.625 μM (fucoxanthin and fucoxanthinol) or 10, 5, and 2.5 μM (β-carotene and astaxanthin), followed by incubation at 37° C. for 24 hours. After adding “WST-8” (manufactured by Wako Pure Chemical Industries, Ltd.) in an amount of 5 μL/well as a coloring substrate, cells were cultured for four hours at 37° C. After culturing, absorbance at a wavelength of 450 nm was measured by a microplate reader to determine the survival rate of the cells using the following formula.

Cell survival rate(%)=[1−(A−B)/A]×100 [Formula 1]

A: Absorbance without agent treatment

A: Absorbance with agent treatment

(Results)

The effects of fucoxanthin and fucoxanthinol on proliferation potency of HTLV-I-infected T-lymph cell lines or EBV-infected B cell lines are shown in FIG. 1 and FIG. 2. As is clear from these Figures, fucoxanthin and fucoxanthinol concentration-dependently suppressed the growth of all the HTLV-I-infected T-lymph cell lines and EBV-infected BL cell lines. In addition, the capability of fucoxanthinol to inhibit growth of these cell lines was found to be significantly higher than that of fucoxanthin.

On the other hand, the results of the similar experiment in which the effects of β-carotene and astaxanthin on proliferation potency of HTLV-I-infected T-lymph cell lines were investigated are shown in FIG. 3. As can be seen from the results, other xanthines have only a slight effect on the growth of HTLV-I-infected T-lymph cell lines, whereas the fucoxanthins have an excellent growth inhibiting capability.

### Example 2

### Measurement of Proliferation Potency of Peripheral Blood Mononuclear Cells in Healthy Person and Adult T-Cell-Leukemia (ATL) Patients

### (Method)

First, the peripheral blood mononuclear cells (PBMC) were separated by the Ficoll centrifugal specific gravity method. The cells were diluted with an RPMI 1640 culture medium containing 10% fetal bovine serum to 2×10⁶ cells/mL and spread over a 96-well plate in an amount of 1×10⁵ cells/well.

Next, 50 μL/well of fucoxanthin and fucoxanthinol were added to make final concentrations of 10, 5, 2.5, 1.25, and 0.625 μM (fucoxanthin) or 2.5, 1.25 and 0.625 μM (fucoxanthinol), followed by incubation at 37° C. for 24 hours. After adding WST-8 in an amount of 5 μL/well, the cells were cultured for four hours at 37° C. After culturing, absorbance at a wavelength of 450 nm was measured by a microplate reader to determine the survival rate of the cells using the above formula.

(Results)

The effects of fucoxanthin and fucoxanthinol on proliferation potency of the PBMC of a healthy person and five adult T-cell-leukemia (ATL) patients are shown in FIG. 4. As is clear from the figure, fucoxanthin and fucoxanthinol concentration-dependently suppressed the growth of leukemia cells in the ATL patients, but exhibited no toxicity against the PBMC of the healthy person. The effect of fucoxanthinol on growth potency was higher than that of fucoxanthin.

### Example 3

### Measurement of Cell-Cycle of HTLV-I-Infected Cell Lines

### (Method)

1×10⁶ cells of HTLV-I-infected cell lines (MT-2, MT-4, HUT-102, ED-40515 (−)) were scattered over a cell culture plate, and 5 μM of fucoxanthin was added, followed by incubation at 37° C. for 24 hours. After 24 hours, the cells were collected and dyed with propidium iodide to measure the DNA content using a flow sight meter. The cell cycle of each cell was judged by calculating the distribution of the cell group of each cell cycle from the result of the DNA content.

(Results)

The effect of fucoxanthin on the cell cycle of the HTLV-I-infected cell lines is shown in FIG. 5.

As a result, the cell groups were found to have increased in the GI phase, confirming that fucoxanthin suspends the cell cycle of all HTLV-I-infected cell lines at the G1 phase.

### Example 4

### Measurement of Apoptosis HTLV-I-Infected Cell Lines

### (Method)

1×10⁶ cells of HTLV-I-infected cell lines (MT-2, MT-4, HUT-102, ED-40515 (−)) were scattered over a cell culture plate, and fucoxanthin (10, 5, and 2.5 μM) or fucoxanthinol (10, 5.25, 1.25, and 0.625 μM) was added, followed by incubation at 37° C. for 24 hours. After 24 hours, the cells were collected and dyed with annexin V to measure the rate (%) of apoptosis-positive cells using a flow sight meter.

(Results)

The effect of fucoxanthinol on apoptosis of HTLV-I-infected cells is shown in FIG. 6 and the relationships between the concentration of fucoxanthin and fucoxanthinol with the apoptosis of MT-2 are shown in FIGS. 7 and 8 respectively. It can be seen from the results that fucoxanthin induces apoptosis in all HTLV-I infected cell lines (FIG. 6) and that the effect is concentration dependent (FIG. 7). It was also confirmed that fucoxanthinol induces apoptosis in MT-2, one of the HTLV-I-infected cell lines, and that the effect is stronger than that of fucoxanthin (FIG. 8).

### INDUSTRIAL APPLICABILITY

As shown in the above Examples, fucoxanthin and fucoxanthinol concentration-dependently reduce the survival rate of malignancy related to viruses such as HTLV-I-infection T-lymph cell lines, EBV-infected BL cell lines, EBV-infected B cell lines, and ATL cells, without affecting the peripheral blood mononuclear leukocytes of healthy persons.

Therefore, the therapeutic agent for virus-associated malignancy of the present invention has a selective anti-tumor effect on viral infected lymphocytes and can be used as a practically applicable novel agent for treating or preventing ATL, Burkitt lymphoma, and the like.

### BRIEF DESCRIPTION OF THE DRAWINGS

FIG. 1 shows effects of fucoxanthin and fucoxanthinol on proliferation potency of HTLV-I-infected T-lymph cell lines, wherein A shows the effect of fucoxanthin and B shows the effect of fucoxanthinol.

FIG. 2 shows effects of fucoxanthin and fucoxanthinol on proliferation potency of EBV-infected B cell lines, wherein A shows the effect of fucoxanthin and B shows the effect of fucoxanthinol.

FIG. 3 shows effects of β-carotene and astaxanthin on proliferation potency of HTLV-I-infected T-lymph cell lines, wherein A shows the effect of β-carotene and B shows the effect of astaxanthin.

FIG. 4 shows effects of fucoxanthin and fucoxanthinol on the survival rate of peripheral blood mononuclear leukocytes of healthy persons and ATL patients, wherein A shows the effect of fucoxanthin and B shows the effect of fucoxanthinol.

FIG. 5 shows the effect of fucoxanthin on the cell cycle of HTLV-I-infected cell lines.

FIG. 6 shows the effect of fucoxanthin on the apoptosis of HTLV-I-infected cell lines.

FIG. 7 shows the concentration-dependent effect of fucoxanthin on the apoptosis of MT-2, which is one of the HTLV-I-infected cell lines.

FIG. 8 shows the concentration-dependent effect of fucoxanthinol on the apoptosis of MT-2, which is one of the HTLV-I-infected cell lines.

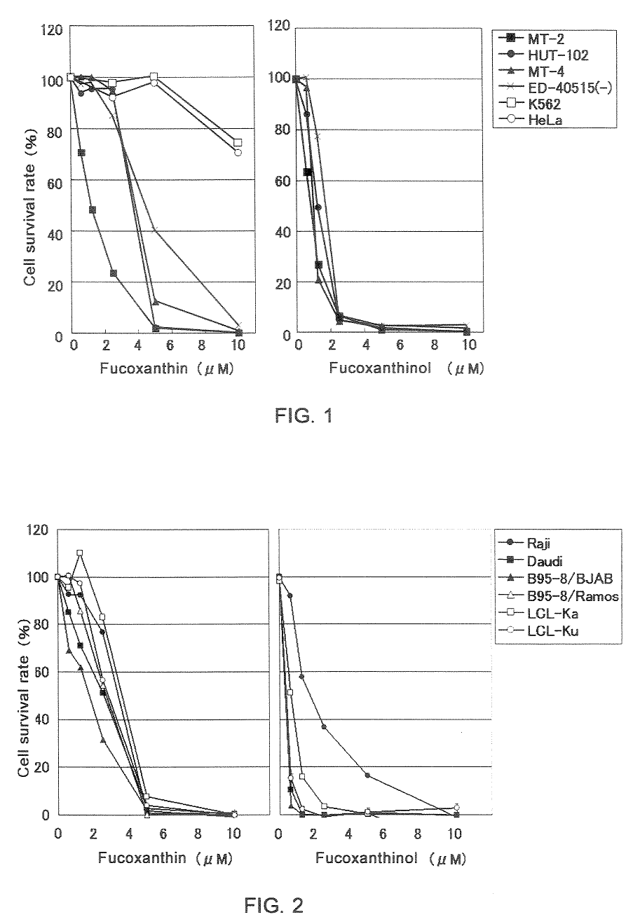

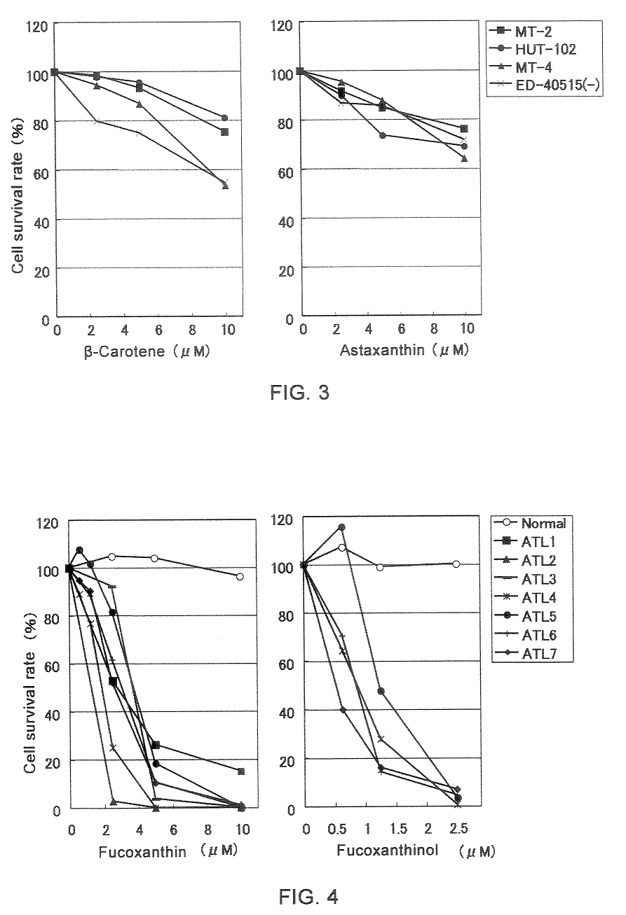

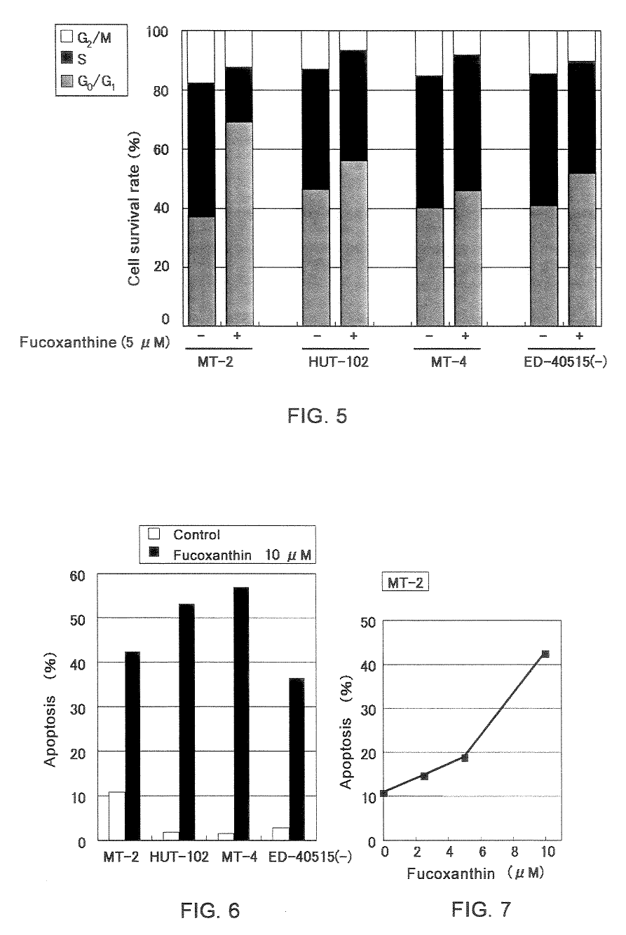

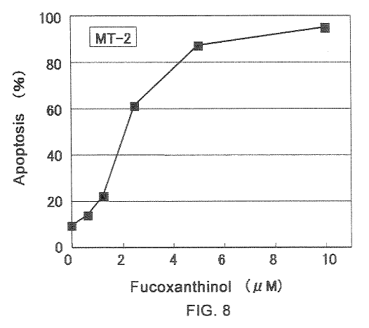

1. A therapeutic agent for virus-associated malignancy comprising fucoxanthin or fucoxanthinol as an active component.

2. The therapeutic agent according to claim 1, for treating or preventing adult T-cell leukemia or Burkitt lymphoma.

3. The therapeutic agent according to claim 1, wherein the agent is a preparation to be orally administered.

4. The therapeutic agent according to claim 1, wherein the agent is a preparation to be parenterally administered.

5. The therapeutic agent according to claim 2, wherein the agent is a preparation to be orally administered.

6. The therapeutic agent according to claim 2, wherein the agent is a preparation to be parenterally administered.

7. A method of treating virus-associated malignancy comprising administering the therapeutic agent according to claim 1 to a patient of the virus-associated malignancy.
